(19) United States
(12) Patent Application Publication
(10) Pub. No.: US 2025/0000428 A1
(43) Pub. Date: Jan. 2, 2025

(54) COMPUTER-IMPLEMENTED METHOD FOR ASSISTING A GENERAL ANESTHESIA OF A SUBJECT

(71) Applicants: PARIS SCIENCES ET LETTRES, PARIS (FR); CENTRE NATIONAL DE LA RECHERCHE SCIENTIFIQUE CNRS, PARIS (FR); NATIONAL INSTITUTE OF HEALTH AND MEDICAL RESEARCH, PARIS (FR); ASSISTANCE PUBLIQUE HÔPITAUX DE PARIS, PARIS (FR)

(72) Inventors: David HOLCMAN, PARIS (FR); Matteo DORA, PARIS (FR); Christophe SUN, PARIS (FR)

(73) Assignees: PARIS SCIENCES ET LETTRES, PARIS (FR); CENTRE NATIONAL DE LA RECHERCHE SCIENTIFIQUE CNRS, PARIS (FR); NATIONAL INSTITUTE OF HEALTH AND MEDICAL RESEARCH, PARIS (FR); ASSISTANCE PUBLIQUE HÔPITAUX DE PARIS, PARIS (FR)

(21) Appl. No.: 18/292,783

(22) PCT Filed: Jul. 26, 2022
(86) PCT No.: PCT/EP2022/070886

§ 371 Date: Jan. 26, 2024

(30) Foreign Application Priority Data
Jul. 28, 2021 (EP) 21306053.6

(51) Int. Cl. A61B 5/374; A61B 5/00

(52) U.S. Cl. CPC A61B 5/374; A61B 5/4821; A61B 5/7203; A61B 5/726; A61B 5/7282

(57) ABSTRACT

A computer-implemented method for assisting a general anesthesia or a sedation of a subject, having
receiving an electroencephalogram EEG signal of a subject,
processing the EEG signal including α-bands for
   defining for each instant of a plurality of instants, a part of the signal has in a time window having the instant, the part of the signal being related to the instant,
   determining for each instant, a distribution of a power of the part of the signal as a function of the frequency,
   determining for each instant a maximum power frequency as a frequency of maximum power in the distribution, the maximum power frequency being related to the instant,
   detecting a reference instant, so that the maximum power frequency as a function of time takes higher values before the reference instant than after, detecting in a time frame following the reference instant, an alpha instant corresponding to a first occurrence of a suppression of an α-band, estimating a first parameter as the duration between the reference instant and the alpha instant, the first parameter relating to a sensitivity of the subject to a general anesthesia or sedation.

### FIELD OF THE INVENTION

The present invention relates to the field of method for assisting a general anesthesia of a subject and in particular electroencephalogram (EEG) signal analysis in the specific context of general anesthesia (GA).

### BACKGROUND OF THE INVENTION

General anesthesia is a medically induced coma with loss of protective reflexes, resulting from the administration of one or more anesthetic agents. It is carried out to allow medical procedures that would otherwise be intolerably painful for the subject, or where the nature of the procedure itself precludes the patient being awake. In order to be in control of a general anesthesia, the literature mainly provides a fist approach consisting in signal processing to analyze real time events such as electroencephalogram (EEG) signal or a second approach consisting in measuring and estimating the effect of parameters such as the age, the arterial pressure, etc.

Monitoring a general anesthesia is currently based on the use of EEG, which is easy and inexpensive to acquire. EEG is the benchmark for monitoring intraoperative brain activity.

This monitoring is mainly focused on the depth of anesthesia (DoA), defined as the degree of central nervous system depression produced by anesthetics.

Commercial DoA monitors can reveal various characteristics determined from the EEG signal, such as percentage of fast and slow wave or the degree of wave synchronization.

Various frequency bands can be extracted and also transient patterns such as iso-electrical suppressions (IES) that consist of periods of iso-electric (flat) activity lasting from few seconds to several minutes. These suppressions appear in several pathological conditions such as epileptic encephalopathies, drug intoxications, comatose or brain death. They are also associated with post-operative sleep disorder, the emergence of delirium, and an increased mortality in sedated critically ill persons. IES can also appear when the concentrations of anesthetics increase.

It is therefore a recommendation to avoid anesthetic drug-related IES during anesthesia.

If commercial DoA monitors can detect IES, those tools cannot predict the occurrence of IES.

More generally, there is no robust method to anticipate and prevent IES, and no method to predict the sensitivity of a subject to general anesthesia.

Being able to predict IES sufficiently in advance with a high accuracy make possible to act on the doses of anesthetic administered, for example by reducing them.

There is thus a need to provide for predictive information on the sensitivity of a subject to general anesthesia and the risk of IES due to general anesthesia.

### BRIEF DESCRIPTION OF THE INVENTION

It is an object of this invention to provide a method for estimating a sensitivity of a subject to general anesthesia or sedation that allows to act on the doses of anesthetic administered.

To that end, the invention provides a computer-implemented method for assisting a general anesthesia or a sedation of a subject, comprising:
receiving an electroencephalogram EEG signal of a subject,
processing the EEG signal comprising α-bands for
   defining for each instant of a plurality of instants, a part of the signal comprised in a time window comprising the instant, the part of the signal being related to the instant,
   determining for each instant, a distribution of a power of the part of the signal as a function of the frequency,
   determining for each instant a maximum power frequency as a frequency of maximum power in the distribution, the maximum power frequency being related to the instant,
   detecting a reference instant, so that the frequency of maximal power takes higher values before the reference instant than after,
   detecting in a time frame following the first instant, an alpha instant corresponding to a first occurrence of a suppression of an α-band,
estimating a first parameter as the duration between the reference instant and the alpha instant, said first parameter relating to a sensitivity of the subject to a general anesthesia.

First the alpha duration can be extracted in the first minutes following the loss of consciousness of the general anesthesia, that is significantly before the administration of doses of anesthetic for the maintenance phase.

Second, the alpha duration carries relevant information with regards to iso-electrical suppressions (IES) the subject may experience in the close future.

This method thus provides predictive information on the sensitivity of a subject to general anesthesia sufficiently in advance to possibly modify doses of anesthetic to be administered.

Such method is advantageously and optionally completed by the following features taken singly or in combination:
detecting time spans in the signal, a suppression of an α-band extending on each time span,
estimating a second parameter as a rate of increase of a proportion of the time spans in the signal, said second parameter relating to a sensitivity of the subject to a general anesthesia or a sedation;
detecting in a time frame following the reference instant, an iso-electric instant of a first occurrence of an iso-electric suppression,
estimating a third parameter as the duration between the reference instant and the iso-electric instant, said third parameter relating to a sensitivity of the subject to a general anesthesia or a sedation;
determining an induction instant following a predetermined induction duration after the reference instant,
determining a final instant following a predetermined final duration after the induction instant,
if the alpha instant is before the induction instant and the iso-electric instant, determining at the alpha instant the first parameter,
if the iso-electric instant is before the induction instant and the alpha instant, determining at the iso-electric instant the third parameter,
determining a first estimate of the second parameter at the induction instant,
determining at the final instant, a second estimate of the second parameter, the first parameter if the alpha instant is before the final instant and the third parameter if the iso-electric instant is before the final instant;
detecting the reference instant comprises
   fitting a curve of the frequency of maximal power as a function of time using a sigmoid function,
   determining a range of values and a final value of the sigmoid function,
determining the reference instant as an instant when the sigmoid function reaches an intermediate value, a difference between the intermediate value and the final value being equal to 5% of the range of values;
detecting an artifact signal within the signal,
determining a ground signal using a portion of the signal immediately before or after the artifact,
decomposing the artifact signal and respectively the ground signal using a discrete wavelet transform so as to determine, for each level of the transform an artifact projection of the artifact signal and respectively a ground projection of the ground signal, the artifact projection and respectively the ground projection being a projection of the artifact signal respectively the ground signal on a basis of the discrete wavelet transform,
for each level m of the transform determining an artifact cumulative density function

$${F}_{m}^{( art )} ( x ) = \frac{1}{{N}_{m}} {\sum }_{n = 1}^{{N}_{m}} {1}_{\vert {w}_{m , n}^{( art )} \vert < x}$$

where Nₘ is the total number of coefficients of the artifact projection at level m, |w_{m,n}^{(art)}| is the absolute value of a n-th coefficient of the artifact projection at level m, and 1_{|w}_{_{m,n}}_{^{(art)}}_{|<x} is an indicator function that returns the value 1 if |w_{m,n}^{(art)}|<x and else the value 0,
for each level m of the transform determining a ground cumulative density function

$${F}_{m}^{( ref )} ( x ) = \frac{1}{{N}_{m}} {\sum }_{n = 1}^{{N}_{m}} {1}_{\vert {w}_{m , n}^{( ref )} \vert < x}$$

where Nₘ is the total number of coefficients of the artifact projection at level m, |w_{m,n}^{(ref)}| is the absolute value of a n-th coefficient of the artifact projection at level m, and 1_{|w}_{_{m,n}}_{^{(ref)}}_{|<x} is an indicator function that returns the value 1 if |w_{m,n}^{(ref)}|<x and else the value 0,
for each level m and for each artifact coefficient w_{m,n}^{(art)} determining a corrected coefficient w_{m,n}^{(corr)} of a corrected projection, the artifact coefficient and the corrected coefficient being similarly indexed, following the sub-steps:
   determining the value Fₘ^{(art)}(|w_{m,n}^{(art)}|),
   determining an intermediate value x verifying Fₘ^{(ref)}(x)=Fₘ^{(art)}(|w_{m,n}^{(art)}|),
   setting the corrected coefficient w_{m,n}^{(corr)} equal to

$${w}_{m , n}^{( art )} \times min \{ 1 , \frac{x}{\vert {w}_{m , n}^{( art )} \vert} \} ,$$

determining a corrected signal using the discrete wavelet transform based on the corrected projection,
replacing in the signal the artifact signal by the corrected signal;
a determination of a minimum quantity of anesthetic to inject so that the subject remains in general anesthesia, the determination being based on the first parameter.

It is also an object of this invention to provide a general anesthesia or sedation assisting device comprising a processor configured for implementing the computer implemented method as described above.

It is another object of this invention to provide a computer-readable medium comprising computer-executable instructions embodied which, when executed by a computer, cause the computer to carry out the as described above.

### BRIEF DESCRIPTION OF THE DRAWINGS

Further features and advantages of the invention will be apparent from the following description, based on the appended drawings wherein:

FIG. 1 represents a general anesthesia or sedation assisting device according to an embodiment of the invention;

FIG. 2 represents an electroencephalogram signal;

FIG. 3 represents a spectrogram of an electroencephalogram signal;

FIG. 4 represents a frequency of maximum power fₘₐₓ(t) of an electroencephalogram signal as a function of time;

FIG. 5 represents a curve fitting of the frequency of maximum power fₘₐₓ(t) of FIG. 4;

FIG. 6 represents an electroencephalogram signal comprising suppressions of an α-band and iso-electric suppressions;

FIG. 7 represents a fraction of the signal occupied by suppressions of an α-band, the fraction being a function of time;

FIG. 8 represents ROC curves;

FIG. 9 represents a decision tree method;

FIG. 10 represents artifacts in an electroencephalogram signal;

FIG. 11 represents a cumulative function defined through a wavelet decomposition of an electroencephalogram signal;

FIG. 12 represents a separation of two groups of persons with regards of parameters extracted from an electroencephalogram signal.

Reference signs that are identical from one figure to another one designates the same element, or elements fulfilling the same function.

### DETAILED DESCRIPTION OF EMBODIMENTS OF THE INVENTION

### General Anesthesia or Sedation Assisting Device

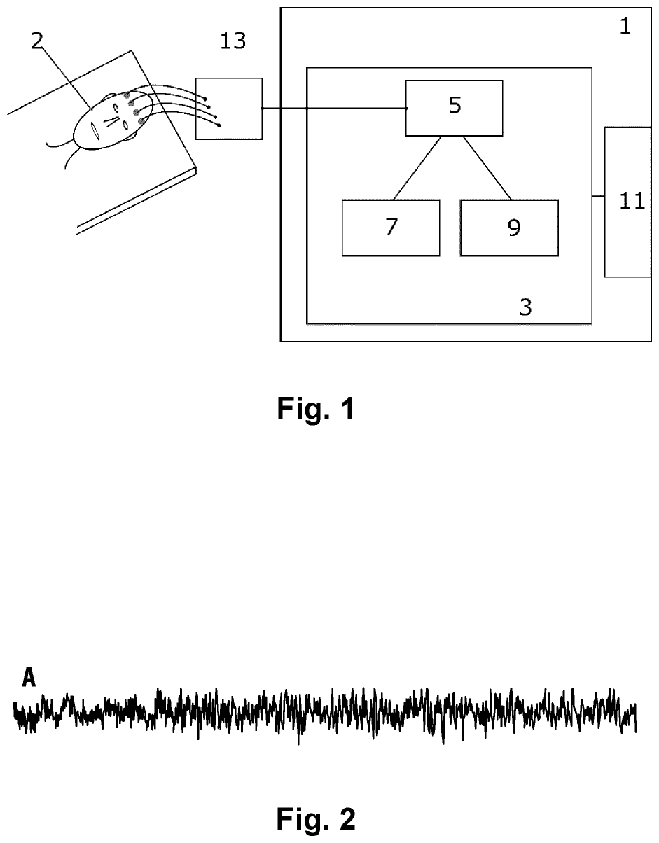

In reference to FIG. 1, a general anesthesia or sedation assisting device is now presented.

According to the invention, the terms “subject” and “patient” are used interchangeably herein and refer to a mammal or several mammals in need of a general anesthesia or a sedation procedure. Subjects are preferably humans or a person.

A person 2 carries electrodes of an electroencephalogram EEG acquisition device 13. The acquisition device 13 is configured to produce an electroencephalogram signal from the person being tested. The acquisition device 13 is connected to a general anesthesia or sedation assisting device 1 that comprises a processor 3 configured to receive and process an electroencephalogram EEG signal.

### Frequency Analysis

The processor 3 comprises a frequency analyzer unit 5 configured to receive the electroencephalogram EEG signal and extract frequency parameters related to the signal. In particular, the frequency analyzer 5 is configured to define a sliding time window and determine a truncated signal equal to the EEG signal within the sliding time window and equal to zero outside the sliding time window. The frequency analyzer is configured to determine a distribution of a power of the truncated signal as a function of the frequency and extract a frequency of maximum power fₘₐₓ. As the time window is centered on an instant, the frequency of maximum power corresponds to an instant. The frequency analyzer 5 can thus produce a spectrogram of the EEG signal and extracts the frequency of maximum power fₘₐₓ(t) of the signal as a function of time.

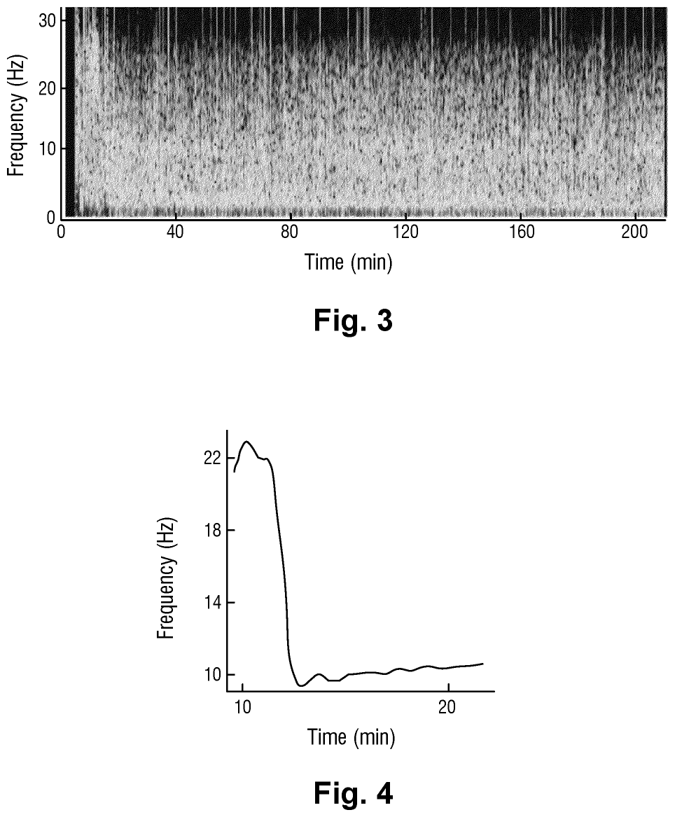

FIG. 3 illustrates an example of a spectrogram of an EEG signal.

FIG. 4 illustrates an example of the frequency of maximum power fₘₐₓ(t) of the signal as a function of time.

The processor 3 is configured to detect a reference instant, so that a function of time, for instance the frequency of maximal power fₘₐₓ(t), takes higher values before the reference instant than after.

### EEG Pattern: Suppression of an α-Band and Iso-Electric Suppression

The processor comprises also a memory 7 in which particular EEG patterns are recorded or predetermined threshold related to particular EEG patterns. In particular EEG patterns without an α-band and EEG patterns of iso-electric suppressions are recorded or predetermined threshold related to particular EEG patterns without an α-band and EEG patterns of iso-electric suppressions are recorded.

The processor 3 is configured to identify a suppression of an α-band, further named as, within the signal and timestamp the suppression.

For this purpose, different methods may be implemented by the processor 3.

A first option consists in filtering the signal in the alpha band and checking that the amplitude is below a predetermined threshold related to particular EEG patterns without an α-band and further in filtering the signal in the delta band and checking that the filtered signal presents a power above a predetermined power.

A second option consists in comparing the signal to particular EEG patterns without an α-band and to particular EEG patterns of iso-electric suppressions and according to the similarities detected deciding if the signal compared corresponds to a suppression of an α-band.

Simple indicators and simple filtering tools very common in the field are sufficient to make this identification of a suppression of an α-band within the signal and timestamp of the suppression.

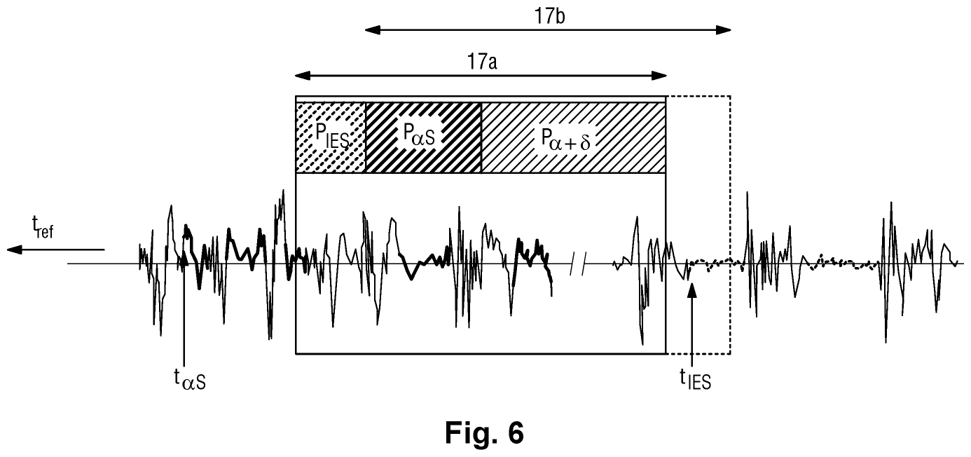

FIG. 6 illustrates an example of an EEG signal. Only a part of the EEG signal is illustrated on FIG. 6. The processor is configured to detect suppressions of an α-band within the signal. The first suppression is illustrated on FIG. 6 and occurs at the alpha instant t_{αS}.

The processor 3 may optionally be further configured to detect time spans in the signal, a suppression of an α-band or αS extending on each time span. The processor is then configured to estimate a rate of increase of a proportion of the time spans in the signal. In other words, the processor defines sliding time windows in the signal and estimate the fraction of the time window that is occupied by the time spans. The duration of the sliding time window may be chosen equal to 240 seconds. The analysis of the sliding time window may be configured to analyze the content of time windows temporally separated with a delay of 1 second, so that two successive time windows present an overlap of 239 seconds.

FIG. 6 illustrates a first time window 17a and a second time window 17b, the second time window 17b starting later than the first time window 17a. Time windows 17a and 17b present an overlap. Within the first time window 17a, the processor counts the time spans and estimates the fraction P_{αS} of the first time window 17a occupied by the time spans.

The time windows may also be occupied by periods of iso-electric suppressions and periods comprising Delta band in the range [0-4] Hz and an Alpha band in the range [8-14] Hz. FIG. 6 illustrates a possible composition of the first time window 17a, that comprises:
a fraction P_{αS} of suppression of an α-band,
a fraction P_{IES} of iso-electric suppression,
a fraction P_{α+δ} of Delta band and Alpha band.

As the time window is centered on an instant, the fraction corresponds to an instant. The processor 3 can thus produce a fraction of the time window that is occupied by the time spans as a function of time. The processor 3 is also configured to determine a rate of increase of this fraction, or, put in another way, a proportion of the time spans in the signal.

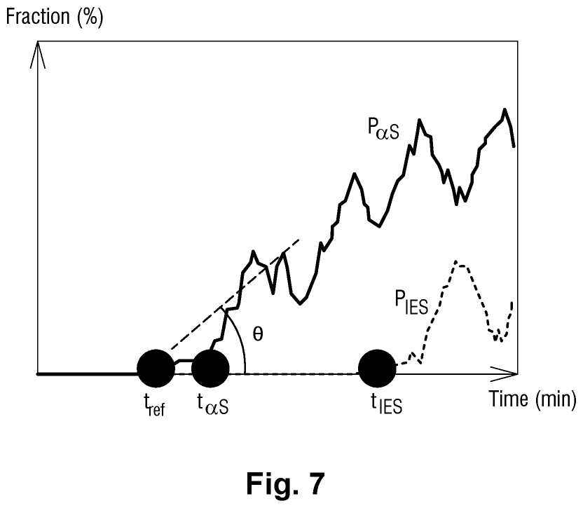

FIG. 7 illustrates an example of a determination of a rate of increase of the fraction P_{αS} of suppression of an α-band. The instants t_{ref} and t_{αS} are represented on the abscises axis. On the ordinate axis, two fractions are represented: the fraction P_{αS} of suppression of an α-band and the fraction P_{IES} of iso-electric suppression.

P_{αS} is equal to zero for a time window temporally located before t_{αS} and takes positive values for a time window that comprises t_{αS} or that is temporally located after t_{αS}. As more suppressions of an α-band occur in the signal, P_{αS} takes higher values. The curve of P_{αS} as a function of time can be linearly fitted by the processor so as to extract a rate of increase tan (θ) or equivalently a slope a.

The processor 3 is configured to identify an iso-electric suppression, further named IES, within the signal and timestamp the suppression.

A first iso-electric suppression is illustrated on FIG. 6 and occurs at the iso-electric instant t_{IES}.

### Sigmoid Function

A model of a sigmoid function is recorded in the memory 7 as follows

$$S ( t ) = \frac{a}{1 + exp ( c ( t - {t}_{0} ) )} + b$$

where a, b, c and t₀ are parameters to be estimated.

The processor 3 is configured to fit a curve C(t) as a function of time using the sigmoid function. Parameters a, b, c and t₀ are found in the process of fitting as parameters minimizing a quantity ∥S(t)−C(t)∥_{L}_{²}_{([0,T}_{₀}_{])}, or put in another form:

{â,\hat{b},ĉ,t_{\hat{0}}}=argmin_{a,b,c,t}_{₀}∥S(t)−C(t)∥_{L}_{²}_{[0,T}_{₀}_{])}

In particular, the curve C(t) can be chosen as the frequency of maximum power of the signal fₘₐₓ(t). Such a fitting procedure allows to determine the portion of the spectrogram, where the beta band disappears and the alpha bands emerges, involving the [8-30] Hz frequencies.

The processor 3 is configured to determine an initial value of the sigmoid function Sₘₐₓ=a+b, a final value of the sigmoid function Sₘᵢₙ=a and a range of values of the sigmoid function Sₘₐₓ−Sₘᵢₙ=b.

The processor 3 is configured to determine an instant t* when the sigmoid function reaches an intermediate value, a difference between the intermediate value and the final value being equal to 5% of the range of values:

$$t * \frac{1}{c} ln ( \frac{1}{0.05} - 1 ) + {t}_{0} .$$

Such an instant t* may be used as a reference instant, so that the sigmoid function takes higher values before the reference instant t* than after.

Other tools that do not involve a sigmoid function may be implemented to determine a reference instant based on the provided maximum power frequency as a function of time, so that the maximum power frequency as a function of time takes higher values before the reference instant than after. For instance, the person skilled in the art may use:
1—an algebraic function of the type

$$a + \frac{1}{( b + c \times {t}^{α} )}$$

or an exponential function of the type e^{b(t-t}^{₀}⁾—both function of the variable t; a,b,c, α and t₀ being parameters to be estimated—instead of the sigmoid function as described above and following similar steps, or
2—a relative power of alpha band computed with respect to the band [0.1-45] Hz and an estimation of the first time to a threshold.

The processor comprises also a calculator unit 9 configured to determine a first parameter as a duration between a reference instant and an alpha instant, a first occurrence of a suppression of an α-band occurring at the alpha instant.

The calculator unit is also configured to determine a third parameter as a duration between a reference instant and an iso-electric instant, a first occurrence of a suppression of an iso-electric suppression occurring at the iso-electric instant.

### Computer-Implemented Method for Assisting a General Anesthesia or a Sedation of a Person

The general anesthesia or sedation assisting device presented above may be used to implement a computer-implemented method for assisting a general anesthesia or a sedation of a person.

In a first step, an electroencephalogram EEG signal of a person is received by the assisting device. The EEG signal can be transferred from the acquisition device to the assisting device in real time or with a delay. The EEG signal may also be acquired by another acquisition device and later transferred to the assisting device.

FIG. 2 illustrates an example of an EEG signal acquired through one of the frontal electrodes of an acquisition device.

The EEG signal is acquired on a person initially conscious that is put into an artificial coma. Before the induction of general anesthesia or sedation, the spectrogram of the EEG can contain a Beta band in the range [14-30] Hz, that characterizes a normal conscious awakening state. The person loses its consciousness at a reference instant t_{ref} called LOC that stands for Loss of Consciousness. After the reference instant t_{ref}, two main bands appear at low rhythms: a Delta band in the range [0-4] Hz and an Alpha band in the range [8-14] Hz. The LOC may be defined as the instant when the dominant brain state switch from a Beta band to an Alpha band.

The EEG signal comprises α-bands.

In a second step, the assisting device processes the EEG signal, extracts the frequency of maximum power fₘₐₓ(t) of the signal as a function of time, and detects the reference instant t_{ref} defined in the following manner: the maximum power frequency as a function of time takes higher values before the reference instant than after. The assisting device also detects in a time frame following the reference instant, an alpha instant corresponding to a first occurrence of a suppression of an α-band. Thanks to EEG patterns without an α-band recorded in the memory, the processor establishes the alpha instant.

For example, a first suppression of an α-band is illustrated on FIG. 6 and occurs at the alpha instant t_{αS}.

In a third step, the assisting device estimates a first parameter as the duration between the reference instant and the alpha instant. This first parameter relates to a sensitivity of the person to a general anesthesia or sedation.

More specifically, the shorter the duration, the more sensitive is the person to a general anesthesia or sedation.

Following this method, one can determine a parameter relating to the sensitivity of the monitored person to general anesthesia or sedation, as soon as a suppression of an α-band occurs. This can occur less than 10 minutes following the loss of consciousness, and enables to obtain relevant information sooner than in the prior art.

In the case where the EEG signal does not comprise any suppression of the α-band, one can only find a minimal bound on the duration between the reference instant and the alpha instant. It is still possible to estimate the duration and retrieve a relevant information for this person who is in this case appear with no sensitivity or a low sensitivity to general anesthesia or sedation.

Following the estimation of the first parameter, a minimum quantity of anesthetic to inject so that the subject remains in general anesthesia or sedation may be determined based on the first parameter.

The computer-implemented method for assisting a general anesthesia or a sedation of a person may optionally comprise the following steps:
detecting time spans in the signal, a suppression of an α-band extending on each time span,
estimating a second parameter as a rate of increase of a proportion of the time spans in the signal, said second parameter relating to a sensitivity of the person to a general anesthesia or sedation.

More specifically, the greater the rate of increase, the more sensitive the person is to general anesthesia or sedation.

The rate of increase corresponds to the term tan (θ) or the slope a or described previously with regards to the linear fit of the curve of P_{αS} in FIG. 7.

In the case where the EEG signal does not comprise any suppression of the α-band, one can estimate the second parameter as equal to zero.

Following this first option, one can determine a parameter relating to the sensitivity of the monitored person to general anesthesia or sedation, as soon as a plurality of suppressions of an α-band have taken place. One can set a specific duration or induction duration about 10 minutes following the loss of consciousness, and obtain the second parameter relevant with regards of the sensitivity sooner than in the prior art.

The computer-implemented method for assisting a general anesthesia or a sedation of a person may optionally comprise the following steps
detecting in a time frame following the reference instant, an iso-electric instant of a first occurrence of an iso-electric suppression,
estimating a third parameter as the duration between the reference instant and the iso-electric instant, said third parameter relating to a sensitivity of the person to a general anesthesia or sedation.

For example, a first iso-electric suppression is illustrated on FIG. 6 and occurs at the iso-electric instant t_{IES}.

More specifically, the shorter the duration, the more sensitive is the person to a general anesthesia or sedation.

In the case where the EEG signal does not comprise any iso-electric suppression, one can only find a minimal bound on the duration between the reference instant and the iso-electric instant. It is still possible to estimate the third parameter and retrieve a relevant information for this person who is in this case appear with no sensitivity or a low sensitivity to general anesthesia or sedation.

Following this second option, one can determine a parameter relating to the sensitivity of the monitored person to general anesthesia or sedation, as soon as an iso-electric suppression takes place. This can occur less than 10 minutes following the loss of consciousness, and enables to obtain relevant information sooner than in the prior art.

As for the first parameter, a minimum quantity of anesthetic to inject so that the subject remains in general anesthesia or sedation may be determined based on the second or third parameter.

Usually, the iso-electric instant occurs after the alpha instant, but the iso-electric instant can also occur before the alpha instant.

It is thus a possibility to determine the third parameter before or after the first parameter.

To take into account this possibility, an adaptable version of the method is proposed, the adaptable version comprising both first and second options of the process described above. In the adaptable version, the method further comprises the determination of an induction instant following a predetermined induction duration after the reference instant.

The first minutes of anesthesia are currently known as an induction phase.

For instance, the predetermined induction duration can be set equal to 10 minutes, so that the induction instant, t_{ind}, occurs 10 minutes after the reference instant.

The method comprises also the determination of a final instant, t_{fin}, following a predetermined final duration after the induction instant.

For instance, the predetermined final duration can be set equal to 5 minutes, so that the final instant occurs 5 minutes after the induction instant.

The method enables to determine an alpha instant and an iso-electric instant. If the alpha instant is before the induction instant and the iso-electric instant, then the processor determines at the alpha instant the first parameter.

If the iso-electric instant is before the induction instant and the alpha instant, then the processor determines at the iso-electric instant the third parameter.

In all cases, the processor determines a first estimate of the second parameter at the induction instant.

In all cases, the processor determines at the final instant, a second estimate of the second parameter, the first parameter if the alpha instant is before the final instant and the third parameter if the iso-electric instant is before the final instant.

It is possible that the alpha instant is after the induction instant or respectively the final instant, thus only a minimal bound on the duration between the reference instant and the alpha instant can be determined at the induction instant or respectively at the final instant.

Implementing the adaptable version of the method, one can determine as soon as possible a parameter relating to the sensitivity of the monitored person to general anesthesia or sedation: before the induction instant if a suppression of an α-band or an iso-electric suppression occurs before the induction instant, or else at the induction instant. At the final instant, a second estimate of the second parameter, which is the rate of increase tan (θ) or slope a is produced. As this second estimate is based on a longer duration of the EEG signal, the second estimate is more accurate and provides a more relevant information at the final instant.

Optionally to the different implementations of the method described so far, the reference instant may be determined following the steps of:
fitting a curve of the frequency of maximal power as a function of time using a sigmoid function,
determining a range of values and a final value of the sigmoid function,
determining the reference instant as an instant when the sigmoid function reaches an intermediate value, a difference between the intermediate value and the final value being equal to 5% of the range of values.

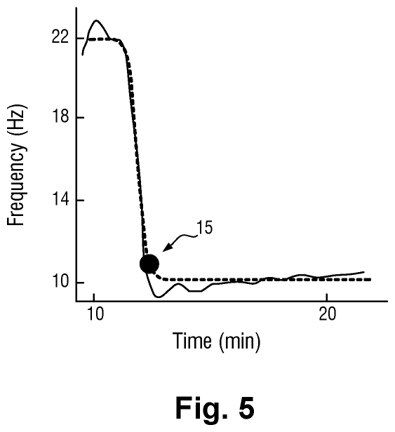

FIG. 5 illustrates an example of the determination of a reference instant 15.

In this example,
the fitted sigmoid follows the expression:

$$S ( t ) = \frac{12.26}{1 + exp ( 5.07 \times ( t - 11.58 ) )} + 10.38$$

and
the reference instant is given by

$$\frac{1}{5.07} ln ( \frac{1}{0.05} - 1 ) + 10.38 \approx 12.26 min .$$

This fitting allows to determine the portion of the spectrogram, where the beta band disappears and the alpha bands emerges, involving the [8-30] Hz frequencies. The result provides the exact location of the time continuous alpha band. This approach is more accurate than in the prior art and may be implemented even when the spectrogram is fragmented or the alpha band is not clearly visible.

### Predictive Value of the Estimated Parameters

The first parameter (t_{αS}−t_{ref}), the second parameter (a) and the third parameter (t_{IES}−t_{ref}) can be used to predict the anesthesia sensitivity of the person and allow to estimate if the person belongs to:
the ‘IES group’ of persons that experience an IES during a general anesthesia or a sedation (that is the persons who have an EEG signal comprising more than 12 s of IES during the first 35 min of anesthesia or sedation), or
the ‘no IES group’ of persons that do not experience an IES during a general anesthesia or a sedation (that is the persons who have an EEG signal comprising less than 12 s of IES during the first 35 min of anesthesia or sedation).

To analyze the predictive value of the three parameters, they are first projected into the three normalized variables:
a logarithm of the normalized alpha instant t_{αS}=log (t_{ind}/(t_{αS}−t_{ref})+1) where t_{ind}=10 min.
a normalized slope a=a/∥a∥_{∞}, where ∥a∥_{∞} is the maximum slope amplitude computed over the EEG recordings,
a logarithm of the normalized iso-electric instant t_{IES}=log (t_{ind}/(t_{IES}−t_{ref})+1) with t_{ind}=10 min.

One can deduce the parameters a, t_{αS}, t_{IES}, from the parameters (t_{αS}−t_{ref}), (a), (t_{IES}−t_{ref}) and vice-versa.

The parameters (t_{αS}−t_{ref}), (a), (t_{IES}−t_{ref}) or equivalently the parameters a, t_{αS}, t_{IES} are further designated as the three parameters.

In a first study, 94 persons experience a general anesthesia and for each one of them the status IES/no IES was established on the one hand and the three parameters were estimated on the other hand. The groups IES/no IES appear well separated between the persons with lower values of t_{αS} and higher values of t_{αS}, and equivalently with respect to the parameters a and t_{IES}.

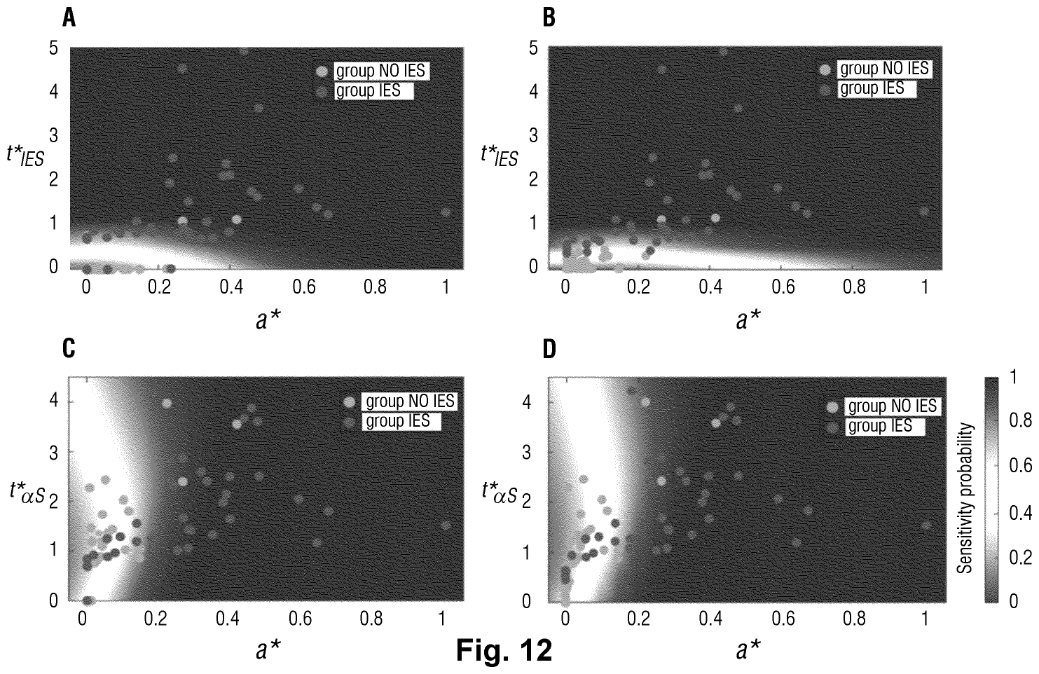

FIG. 12 illustrates the separation of the groups with regards to the couple of parameters a and t_{IES}, and the couple of parameters a and t_{αS}.

Parts A,B of FIG. 12 represents a diagram with the parameter t_{IES}.on the abscises axis and the parameter a on the ordinate axis.

Parts C,D of FIG. 12 represents a diagram with the parameter t_{αS}.on the abscises axis and the parameter a on the ordinate axis.

Parts A, C of FIG. 12 relate to the induction phase, that is the first 15 minutes following the reference instant.

Parts B,D of FIG. 12 relate to the whole general anesthesia.

Each person of the 94 persons is represented on each diagram by a dot. Persons of the “IES group” correspond to a darker spot than the persons of the “No IES group”.

In parts A, B of FIG. 12, persons of the “No IES group” are mostly located to the bottom left of the diagram, whereas persons of the “IES group” occupies the remaining space.

In parts C,D of FIG. 12, persons of the “No IES group” are mostly located to the left of the diagram, whereas persons of the “IES group” occupies the remaining space.

A probability map with different levels of grey is also represented on each diagram. The probability map is deduced from the logistic regression model between the two variables involved in the diagram.

To quantify the predictive value of the three parameters restricted to the induction phase a Monte-Carlo Cross Validation method was implemented. Such a method consists in creating multiple random splits of the patient data under the ratio of 70% training and 30% testing. The number of sampling was set to 50. In each sampling, several logistics models using various combinations of the three parameters are trained and tested. The combinations are compared by computing the mean ROC curves and AUC from these 50 experiments.

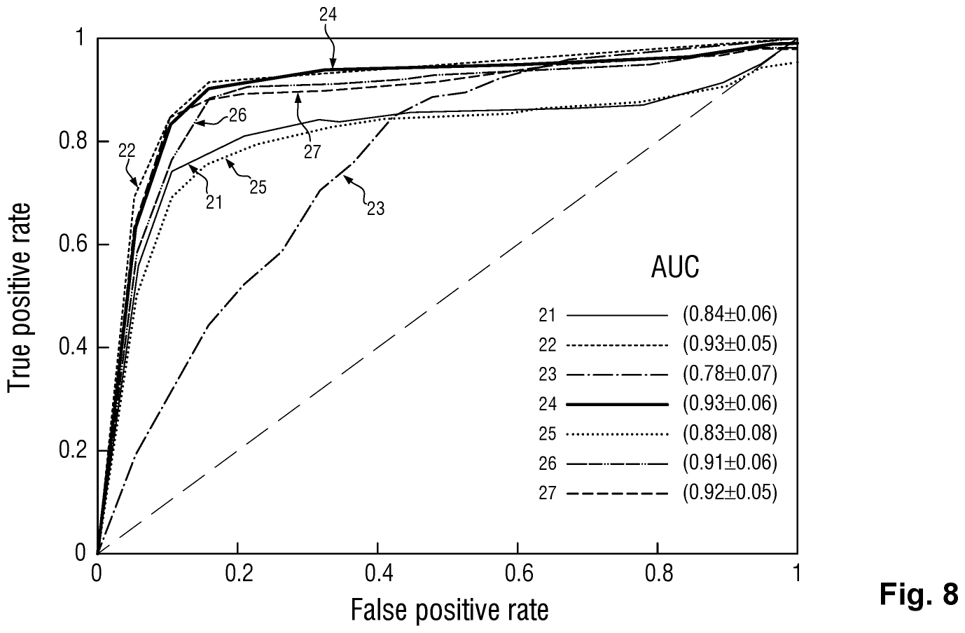

FIG. 8 illustrates the result of this study, based on the 15 first minutes of the anesthesia

The curve 21 corresponds to a logistics model using only the parameter a and correspond to AUC=0.84.

The curve 22 corresponds to a logistics model using only the parameter trEsand correspond to AUC=0.93.

The curve 23 corresponds to a logistics model using only the parameter t_{αS} and correspond to AUC=0.78.

The curve 24 corresponds to a logistics model using both parameters a, t_{IES} and correspond to AUC=0.93.

The curve 25 corresponds to a logistics model using both parameters a, t_{αS} and correspond to AUC=0.83.

The curve 26 corresponds to a logistics model using both parameters t_{αS},ties and correspond to AUC=0.91.

The curve 27 corresponds to a logistics model using the three parameters a, t_{αS}, tIES and correspond to AUC=0.92.

The couple (a, t_{IES}.) appears as the best predictor with an AUC=0.93±0.06, but this couple implies to wait sufficiently to collect the first IES. This result should be compared with the result obtained for the couple (a, t_{αS}) with lower predictive value AUC=0.83±0.08, but this couple can usually be estimated much earlier during the induction phase.

To further validate the predictions within the induction phase, a logistic regression was applied over the similar data, but base on the entire anesthesia time. Very similar ROC curves were obtained, showing that the sensitivity probability computed from the induction phase is robust and does not change during the entire anesthesia.

### Estimating a Sensitivity of a Person to General Anesthesia or Sedation

The three parameters a, t_{αS}, t_{IES} characterize the sensitivity to general anesthesia or sedation and can be collected at different times during the induction phase.

The sensitivity may be expressed as the probability that the monitored person is part of the “IES group”, probability written P(IES=1) or equivalently P(S=1). It is possible to estimate a value of the sensitivity based on the alpha instant t_{αS}, noted P(IES=1|t_{αS}) or equivalently P(S=1|t_{αS}) between the reference moment and the alpha moment, using logistic regression in the following form:

$$P ( IES = 1 \vert {t}_{α S} ) = {( 1 + exp [ - ( γ + {δ}_{0} ( {t}_{α S} ) ) ] )}^{- 1} .$$

It is thus proposed a method for estimating a sensitivity of a subject to general anesthesia or sedation comprising:
receiving an electroencephalogram EEG signal of a subject,
processing the EEG signal comprising α-bands for
   defining for each instant of a plurality of instants, a part of the signal comprised in a time window comprising the instant, the part of the signal being related to the instant,
   determining for each instant, a distribution of a power of the part of the signal as a function of frequency,
   determining for each instant a maximum power frequency as a frequency of maximum power in the distribution, the maximum power frequency being related to the instant,
   detecting a reference instant, so that the maximum power frequency as a function of time takes higher values before the reference instant than after,
   detecting in a time frame following the reference instant, an alpha instant corresponding to a first occurrence of a suppression of an α-band,
estimating a first value of the sensitivity characterizing the risk of IES of the subject put under general anesthesia or sedation based on an alpha duration between the reference moment and the alpha moment.

The sensitivity involved here is the probability P(IES=1|t_{αS}). The following decision rule may be used in order to make medical decisions:
P(IES=1|t_{αS})≥ 0.5 corresponds to the decision «the subject is sensitive to general anesthesia and is part of the group IES »
P(IES=1|t_{αS})<0.5 corresponds to the decision «the subject is not sensitive to general anesthesia and is part of the group No-IES »

This method may be further adapted to estimate other values of the sensitivity of the subject.

For instance, it is possible to estimate also a value of the sensitivity based on the alpha duration t_{αS} and the rate of increase or slope a, noted P(IES=1|t_{αS}, a) using logistic regression in the following form:

$$P ( IES = 1 \vert {t}_{α S} , α ) = {( 1 + exp [ - ( γ + {δ}_{1} ( a ) + {δ}_{2} ( {t}_{α S} ) ) ] )}^{- 1} , \\ with γ = - 1.63 , {δ}_{1} = 14.47 , {δ}_{2} = - 0.27 .$$

It is possible to estimate a value of the sensitivity based on the iso-electric duration t_{IES} and the rate of increase or slope a, P(S=1|t_{IES}, a) using logistic regression in the following form:

$$P ( S = 1 \vert {t}_{IES} , a ) = {( 1 + exp [ - ( γ + {δ}_{1} ( a ) + {δ}_{2} ( {t}_{IES} ) ) ] )}^{- 1} , \\ with γ = - 2.41 , {δ}_{1} = 1.47 , {δ}_{2} = 4.58 .$$

Similar probabilit_{IES} may be introduced such as P(IES=1|a) and P(IES=1|t_{IES}). For all probabilities, the previous decision rule with the 0.5 threshold may be used in order to make medical decisions.

A decision tree procedure may also be implemented in order to quickly retrieve relevant information, make a decision on the status IES/No IES of the subject and define warnings at different instants or checkpoints about the sensitivity.

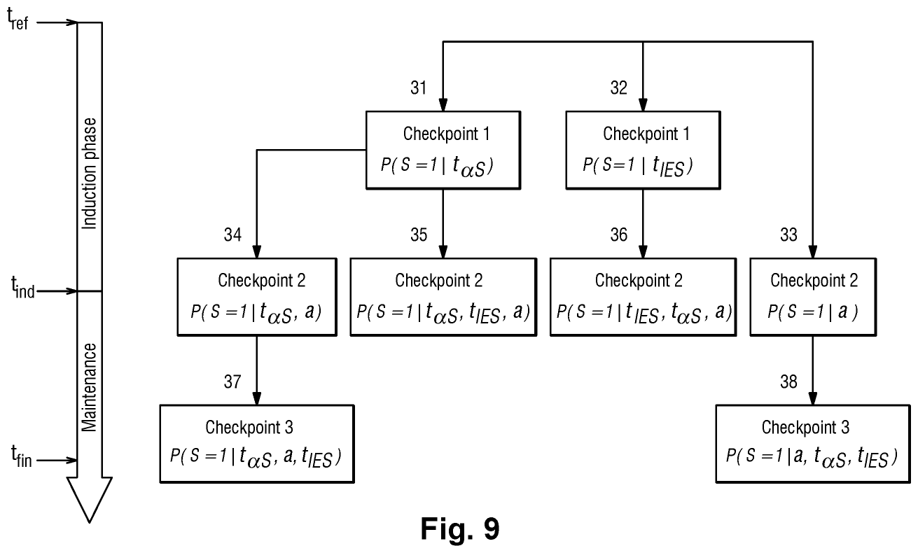

FIG. 9 illustrates schematically the decision tree.

A temporal axis is represented on the left part of the FIG. 9, and the chronological sense is directed downwards of the FIG. 9. The reference instant t_{ref} appears on the top of the axis, above the induction instant t_{ind} and further down appears the final time t_{fin}.

The first checkpoint occurs after the reference instant t_{ref}, and consequently below t_{ref} in FIG. 9.

The first checkpoint corresponds to:
the first time an alpha suppression αS occurs, that is the alpha instant corresponding to the case referenced 31 in FIG. 9, and then an estimate of the sensitivity P(S=1|t_{αS}) can be produced based on the first parameter t_{αS} or equivalenly t_{αS};
the first time an iso-electric suppression IES occurs, that is the iso-electric instant corresponding to the case referenced 32 in FIG. 9, and then an estimate of the sensitivity P(S=1|t_{IES}) can be produced based on the third parameter t_{IES} or equivalenly t_{IES}.

The second checkpoint is set at the induction instant t_{ind}, that is after an induction duration after the reference instant. The second checkpoint is temporally located about the end of the induction phase.

Possibly, no alpha suppression αS and no iso-electric suppression IES occurs before the second checkpoint corresponding to the case referenced 33 in FIG. 9. In this situation, the method does not comprise any first checkpoint. The slope can be estimated to the value zero, and a second estimate of the sensitivity P(S=1|a) can be produced at the second checkpoint.

If the first checkpoint corresponds to the alpha instant, corresponding to the case referenced 31 in FIG. 9, two possible cases can be separated at the second checkpoint.

In a first possible case referenced 34 in FIG. 9, no iso-electric suppression IES occurs before the second checkpoint. In this case, at the second checkpoint, an estimate of the sensitivity P(S=1|t_{αS}, a) can be produced based on the first and second parameters t_{αS}, a.

In the second possible case referenced 35 in FIG. 9, an iso-electric suppression IES occurs before the second checkpoint. In this case, at the second checkpoint, an estimate of the sensitivity P(S=1|t_{αS}, t_{IES}, a) can be produced based on the three parameters.

If the first checkpoint corresponds to the iso-electric instant, corresponding to the case referenced 32 in FIG. 9, an alpha suppression αS occurs before the second checkpoint. In this case referenced 36 in FIG. 9, at the second checkpoint, an estimate of the sensitivity P(S=1|t_{αS}, t_{IES}, a) can be produced based on the three parameters.

Finally, the third checkpoint is involved in the method if the three parameters have not been estimated at the second checkpoint, that is only in the cases referenced 33 and 34 in FIG. 9. For those cases, and if during the final duration between the induction instant and the final instant:
an iso-electric suppression IES occurs in the case referenced 37 following the case 34, an estimate of the sensitivity P(S=1|t_{αS}, t_{IES}, a) can be produced based on the three parameters at the third checkpoint,
an alpha suppression αS and an iso-electric suppression IES occurs in the case referenced 38 following the case 33, an estimate of the sensitivity P(S=1|t_{αS}, t_{IES}, a) can be produced based on the three parameters at the third checkpoint.

Following this decision tree, one can determine and confirm in real time the sensitivity of the monitored person to general anesthesia or sedation. After only 10 and 15 minutes following the loss of consciousness, one can obtain the estimates of the sensitivity.

### Updating a Database for Classification and Parameters Estimation

Once a patient sensitivity is estimated following the decision tree presented above, the sensitivity is then established with certainty by determining the total time the brain has spent in iso-suppression IES at the end of the surgery. If the person has an EEG signal comprising less than 12 s of IES during the first 35 min of anesthesia or sedation, then the person belongs to the ‘no IES group’.

The data of this new person is incorporated in a data basis, and after each monitoring of this type the data basis is completed and updated.

At each new person added in the data base, the settings of the logistic regression may be recomputed. This iterative procedure increases the precision of the prediction each time a person is incorporated in the data base.

In a second aspect of the invention, a process of filtering an EE signal is presented.

Different artifacts can be found in EEG signals.

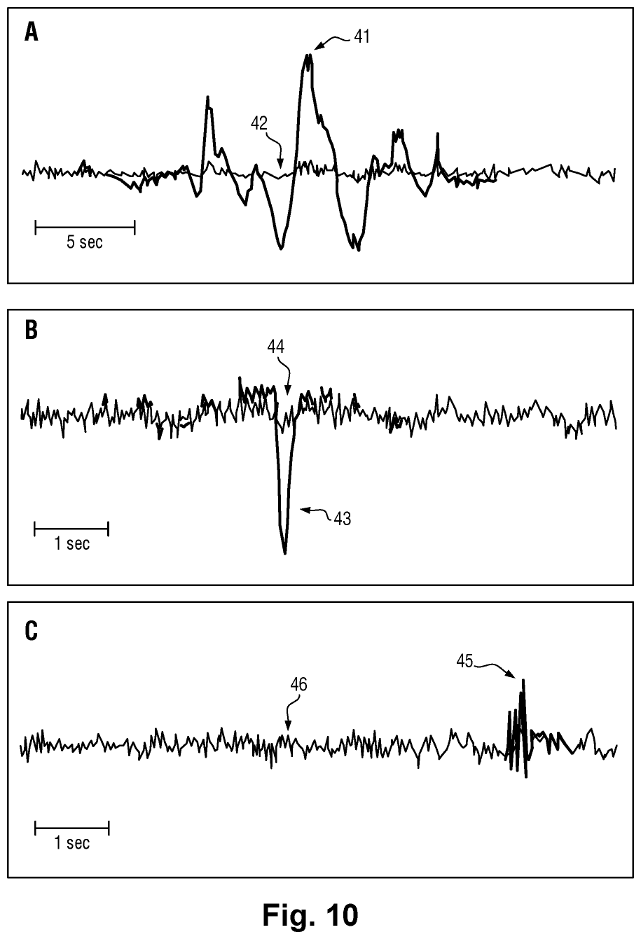

FIG. 10 presents three examples of such artifacts: part A of FIG. 10 illustrates a motion artifact 41, part B of FIG. 10 illustrates an eye blink artifact 43, and part C of FIG. 10 illustrates a muscular artifact 45.

As an artifact appears in the EEG signal, no relevant processing of the signal can be carried out, preventing the anesthetist to retrieve any relevant information in the artifact section.

There is thus a need for a method to remove artifacts in real-time, so that they are replaced by a natural signal and that relevant statistics can be computed.

It is proposed a method to remove artifacts in electroencephalogram signal comprising the following steps:
receiving an electroencephalogram signal of a subject,
detecting an artifact signal within the signal,
determining a ground signal using a portion of the signal immediately before or after the artifact,
decomposing the artifact signal and respectively the ground signal using a discrete wavelet transform so as to determine, for each level of the transform an artifact projection of the artifact signal and respectively a ground projection of the ground signal, the artifact projection and respectively the ground projection being a projection of the artifact signal respectively the ground signal on a basis of the discrete wavelet transform,
for each level m of the transform determining an artifact cumulative density function

$${F}_{m}^{( art )} ( x ) = \frac{1}{{N}_{m}} {\sum }_{n = 1}^{{N}_{m}} {1}_{\vert {w}_{m , n}^{( art )} \vert < x}$$

where Nₘ is the total number of coefficients of the artifact projection at level m, |w_{m,n}^{(art)}| is the absolute value of a n-th coefficient of the artifact projection at level m, and 1_{|w}_{_{m,n}}_{^{(art)}}_{|<x} is an indicator function that returns the value 1 if |w_{m,n}^{(art)}|<x and else the value 0,
for each level m of the transform determining a ground cumulative density function

$${F}_{m}^{( ref )} ( x ) = \frac{1}{{N}_{m}} {\sum }_{n = 1}^{{N}_{m}} {1}_{\vert {w}_{m , n}^{( ref )} \vert < x}$$

where Nₘ is the total number of coefficients of the artifact projection at level m, |w_{m,n}^{(ref)}| is the absolute value of a n-th coefficient of the artifact projection at level m, and 1_{|w}_{_{m,n}}_{^{(ref)}}_{|<x} is an indicator function that returns the value 1 if |w_{m,n}^{(ref)}|<x and else the value 0,
for each level m and for each artifact coefficient w_{m,n}^{(art)} determining a corrected coefficient w_{m,n}^{(corr)} of a corrected projection, the artifact coefficient and the corrected coefficient being similarly indexed, following the sub-steps:
   determining the value Fₘ^{(art)}(|w_{m,n}^{(art)}|),
   determining an intermediate value x verifying Fₘ^{(ref)}(x)=Fₘ^{(art)}(|w_{m,n}^{(art)}|),
   setting the corrected coefficient w_{m,n}^{(corr)} equal to

$${w}_{m , n}^{( art )} \times min \{ 1 , \frac{x}{\vert {w}_{m , n}^{( art )} \vert} \} ,$$

determining a corrected signal using the discrete wavelet transform based on the corrected projection,
replacing in the signal the artifact signal by the corrected signal.

The novel thresholding procedure is an adaptive method using quantile normalization of the wavelet coefficients to attenuate coefficients of the artifact signal.

The use of the cumulative density function enables, for each level of the transform, to compare globally coefficients of the artifact signal and coefficients of the ground signal and to modify the coefficients of the artifact signal so as to set the power of the artifact projection inferior or equal to the power of the ground projection.

Since the distribution of energy across frequency bands for physiological EEG signal should be continuous, no abrupt changes in the power of a band are expected. An artifact is precisely such an abrupt change. Setting the power of the artifact projection equal to the power of the ground projection and defining a corrected signal based on this correction enables to remove the artifacts, replace it by a natural signal so that relevant statistics can be computed.

Also, this method can be implemented in real-time.

The method applies to any EEG signal. In particular, the method may be applied simultaneously with the computer-implemented method for assisting a general anesthesia or a sedation of a subject as described previously.

The invention also concerns a processor configured to implement a method to remove artifacts in electroencephalogram signal as described above.

The general anesthesia or sedation assisting device as described previously comprises a processor that can be also configured to implement the method to remove artifacts in electroencephalogram signal as described above.

The method comprises a first step of receiving an electroencephalogram signal of a subject, this signal can be denoted as the function x(t) of time t.

In a second step, an artifact signal is detected within the signal. This detection can be based on a detection of an abnormal high amplitude signal or by thresholding the signal power to detect non-physiological levels. An artifact time window is thus defined to comprise the detected artifact.

The EEG signal limited to the artifact time window defines an artifact signal x^{(art)}(t),

In a third step, a ground signal is determined using a portion of the signal immediately before or after the artifact. This determination may be implemented by selecting within the EEG signal a ground time window located before the artifact time window and/or after the artifact time window. The ground time window can have the same duration as the artifact window.

The EEG signal limited to the ground time window defines a ground signal x^{(ref)}(t).

In a fourth step, both the artifact signal and the ground signal are decomposed using a discrete wavelet transform so as to determine, for each level of the transform an artifact projection of the artifact signal and a ground projection of the ground signal.

The artifact projection is a projection of the artifact signal on a basis of the discrete wavelet transform.

The ground projection is a projection of the ground signal on a basis of the discrete wavelet transform.

The basis of the discrete wavelet transform consists in:
a first family of functions ψ_{m,n}(t) defined via a first reference function ψ(t) or wavelet function ψ(t), with the relation ψ_{m,n}(t)=2^{=m/2}ψ(2^{-m}t−n),
a second family of functions ϕ_{m,n}(t) defined via a second reference function ϕ(t) or scaling function ϕ(t), with the relation ϕ_{m,n}(t)=2^{-m/2}ϕ(2^{-m}t−n).

A wavelet transform to decompose at level M a signal x(t) into time-frequency components leads to the following expression:

$$x ( t ) = {\sum }_{n} {c}_{M , n} {ϕ}_{M , n} ( t ) + {\sum }_{m = 1}^{M} {\sum }_{n} {d}_{m , n} {ψ}_{m , n} ( t )$$

where m is an integer taking values from 1 to M, n is an integer taking a number Nₘ values depending on m, c_{m,n}= [symbol] x, ϕ_{m,n} [symbol] is the coefficient of projection of x(t) on function ϕ_{m,n}(t) and d_{m,n}= [symbol] x, ψ_{m,n} [symbol] the coefficient of projection of x(t) on function ψ_{m,n}(t).

The scalar product is defined by [symbol] f, g [symbol] =Σ_{t∈Z}f(t)g(t).

The signal x(t) is decomposed onto an orthogonal basis obtained by dilating and translating in time the wavelet function ψ(t) and scaling function ϕ(t).

The decomposition of the artifact signal x^{(art)}(t) produces the artifact coefficients dₘ^{(art)}, c_{M}^{(art)}

The decomposition of the ground signal x^{(ref)}(t) produces the ground coefficients dₘ^{(ref)}, c_{M}^{(ref)}.

To simplify the description, all coefficients can be renamed in the following manner:
w_{m,n}=d_{m,n} for m=1, . . . , M and w_{M+1,n}=c_{M,n} defining: w_{m,n}^{(art)}, w_{m,n}^{(ref)}
w_{m,n} is a notation to designate the coefficients indexed by the integer n: d_{m,n} or c_{M,n}.

In a fifth step, an artifact cumulative density function Fₘ^{(art)}(x) is determined for each level m of the transform with the following definition

$${F}_{m}^{( art )} ( x ) = \frac{1}{{N}_{m}} {\sum }_{n = 1}^{{N}_{m}} {1}_{\vert {w}_{m , n}^{( art )} \vert < x}$$

where Nₘ is the total number of coefficients of the artifact projection at level m, |w_{m,n}^{(art)}| is the absolute value of a n-th coefficient of the artifact projection at level m, and 1_{|w}_{_{m,n}}_{^{(art)}}_{|<x} is an indicator function that returns the value 1 if |w_{m,n}^{(art)}|<x and else the value 0.

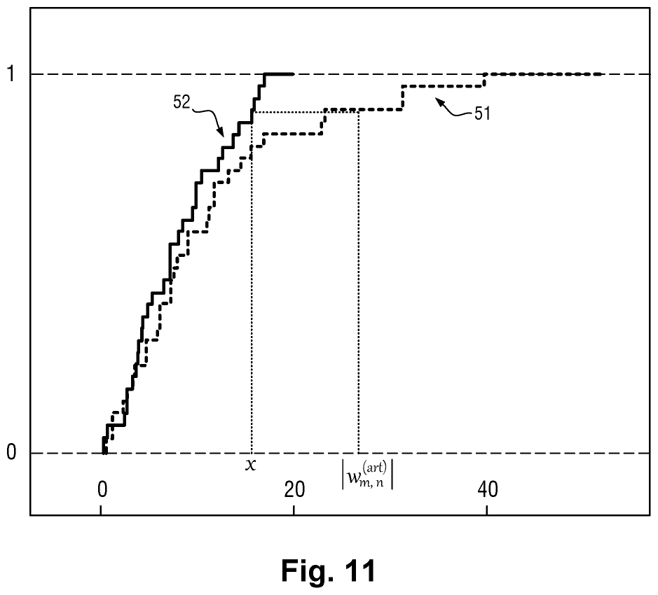

FIG. 11 represents schematically a cumulative density function Fₘ^{(art)}(x) 51 as a function of x. The cumulative density function is a weakly monotonically increasing function taking values between 0 and 1. The cumulative density function takes the value 0 for x=0, and eventually reaches the value 1 for sufficiently high values of x.

In a sixth step, a ground cumulative density function is determined for each level m of the transform with the following definition

$${F}_{m}^{( ref )} ( x ) = \frac{1}{{N}_{m}} {\sum }_{n = 1}^{{N}_{m}} {1}_{\vert {w}_{m , n}^{( ref )} \vert < x}$$

where Nₘ is the total number of coefficients of the artifact projection at level m, |w_{m,n}^{(ref)}| is the absolute value of a n-th coefficient of the artifact projection at level m, and 1_{|w}_{_{m,n}}_{^{(ref)}}_{|<x} is an indicator function that returns the value 1 if |w_{m,n}^{(ref)}|<x and else the value 0.

FIG. 11 represents schematically a cumulative density function Fₘ^{(ref)}(x) 52 as a function of x.

In a seventh step, for each level m and for each artifact coefficient w_{m,n}^{(art)} a corrected coefficient w_{m,n}^{(corr)} of a corrected projection is determined, the artifact coefficient and the corrected coefficient being similarly indexed. This determination follows the sub-steps:
determining the value Fₘ^{(art)}(|w_{m,n}^{(art)}|)—FIG. 11 represents schematically this value;
determining an intermediate value x verifying Fₘ^{(ref)}(x)=Fₘ^{(art)}(|w_{m,n}^{(art)}|)—FIG. 11 represents schematically the intermediate value x;
setting the corrected coefficient w_{m,n}^{(corr)} equal to

$${w}_{m , n}^{( art )} \times min \{ 1 , \frac{x}{\vert {w}_{m , n}^{( art )} \vert} \} ,$$

Using this setting, the artifact coefficient w_{m,n}^{(art)} is globally compared to the entire distribution of ground coefficients.

In an eighth step, a corrected signal x^{(corr)}(t) is determined using the discrete wavelet transform based on the corrected projection. More precisely, the seventh step enables to find a set of corrected coefficients w_{m,n}^{(corr)} in correspondence with the set of artifact coefficients w_{m,n}^{(art)}. d_{m,n}^{(corr)}and c_{m,n}^{(corr)} can thus be defined with the previsously mentioned relations: w_{m,n}=d_{m,n} for m=1, . . . , M and w_{M+1,n}=c_{M,n}.

The corrected signal x^{(corr)}(t) is then determined through the expression

$${x}^{( corr )} ( t ) = {\sum }_{n} {c}_{M , n}^{( corr )} {ϕ}_{M , n} ( t ) + {\sum }_{m = 1}^{M} {\sum }_{n} {d}_{m , n}^{( corr )} {ψ}_{m , n} ( t )$$

Finally, in a ninth step, the artifact signal x^{(art)}(t) is replaced in the signal EEG by the corrected signal x^{(corr)}(t).

The artifact is thus removed from the EEG signal and replaced by a corrected signal which power is inferior or equal to the power of the ground signal neighboring the corrected signal.

Using such a method, the artifacts in FIG. 10 have been removed.

Part A of FIG. 10 illustrates a signal 42 obtained from the EEG that initially comprises the motion artifact 41 and to which the method was applied.

In the same way, part B of FIG. 10 illustrates a signal 44 obtained from the EEG that initially comprises the eye blink artifact 43 and to which the method was applied and part C of FIG. 10 illustrates a signal 46 obtained from the EEG that initially comprises a muscular artifact 45 and to which the method was applied. This method can be implemented in real-time. As the EEG signal is acquired, a corrected EEG signal can be progressively produced using the method.

Advantageously, the presented method enables a reliable monitoring of the EEG signal. When the EEG signal carries an artifact, a spectrogram produced from this signal shows also artifacts. Such noisy perturbations can affect the calculation of spectrogram and the estimates of the depth of anesthesia. The corrected EEG signal may be used to produce a spectrogram with which the parameters relating to the sensitivity of a subject to general anesthesia or sedation can be computed.

1. A computer-implemented method for assisting a general anesthesia or a sedation of a subject, comprising:
receiving an electroencephalogram EEG signal of a subject,
processing the EEG signal comprising α-bands for
defining for each instant of a plurality of instants, a part of the signal comprised in a sliding time window comprising the instant, the sliding time window being centered on the instant, the part of the signal being related to the instant,
determining for each instant, a distribution of a power of the part of the signal as a function of the frequency,
determining for each instant a maximum power frequency as a frequency of maximum power in the distribution, the maximum power frequency being related to the instant,
detecting a reference instant, so that the maximum power frequency as a function of time takes higher values before the reference instant than after,
detecting in a time frame following the reference instant, an alpha instant corresponding to a first occurrence of a suppression of an α-band,
estimating a first parameter as the duration between the reference instant and the alpha instant, said first parameter being further used to assist the general anesthesia or the sedation of the subject, the first parameter relating to a sensitivity of the subject to a general anesthesia or sedation.

2. The method according to claim 1, further comprising:
detecting time spans in the signal, a suppression of an α-band extending on each time span,
estimating a second parameter as a rate of increase of a proportion of the time spans in the signal, said second parameter relating to a sensitivity of the subject to a general anesthesia or sedation.

3. The method according to claim 2, further comprising:
detecting in a time frame following the reference instant, an iso-electric instant of a first occurrence of an iso-electric suppression,
estimating a third parameter as the duration between the reference instant and the iso-electric instant, said third parameter relating to a sensitivity of the subject to a general anesthesia or sedation.

4. The method according to claim 3, comprising:
determining an induction instant following a predetermined induction duration after the reference instant,
determining a final instant following a predetermined final duration after the induction instant,
if the alpha instant is before the induction instant and the iso-electric instant, determining at the alpha instant the first parameter,
if the iso-electric instant is before the induction instant and the alpha instant, determining at the iso-electric instant the third parameter,
determining a first estimate of the second parameter at the induction instant,
determining at the final instant, a second estimate of the second parameter, the first parameter if the alpha instant is before the final instant and the third parameter if the iso-electric instant is before the final instant.

5. The method according to claim 1, wherein:
detecting the reference instant comprises
fitting a curve of the frequency of maximal power as a function of time using a sigmoid function,
determining a range of values and a final value of the sigmoid function,
determining the reference instant as an instant when the sigmoid function reaches an intermediate value, a difference between the intermediate value and the final value being equal to 5% of the range of values.

6. The method according to claim 1, further comprising the following steps:
detecting an artifact signal within the signal,
determining a ground signal using a portion of the signal immediately before or after the artifact,
decomposing the artifact signal and respectively the ground signal using a discrete wavelet transform so as to determine, for each level of the transform an artifact projection of the artifact signal and respectively a ground projection of the ground signal, the artifact projection and respectively the ground projection being a projection of the artifact signal respectively the ground signal on a basis of the discrete wavelet transform,
for each level m of the transform determining an artifact cumulative density function
$${F}_{m}^{( art )} ( x ) = \frac{1}{{N}_{m}} {\sum }_{n = 1}^{{N}_{m}} {1}_{\vert {w}_{m , n}^{( art )} \vert < x}$$
where Nₘ is the total number of coefficients of the artifact projection at level m, |w_{m,n}^{(art)}| is the absolute value of a n-th coefficient of the artifact projection at level m, and 1_{|w}_{_{m,n}}_{^{(art)}}_{|<x} is an indicator function that returns the value 1 if |w_{m,n}^{(art)}|<x and else the value 0,
for each level m of the transform determining a ground cumulative density function
$${F}_{m}^{( ref )} ( x ) = \frac{1}{{N}_{m}} {\sum }_{n = 1}^{{N}_{m}} {1}_{\vert {w}_{m , n}^{( ref )} \vert < x}$$
where Nₘ is the total number of coefficients of the artifact projection at level m, |w_{m,n}^{(ref)}| is the absolute value of a n-th coefficient of the artifact projection at level m, and 1_{|w}_{_{m,n}}_{^{(ref)}}_{|<x} is an indicator function that returns the value 1 if |w_{m,n}^{(art)}|<x and else the value 0,
for each level m and for each artifact coefficient w_{m,n}^{(art)} determining a corrected coefficient w_{m,n}^{(corr)} of a corrected projection, the artifact coefficient and the corrected coefficient being similarly indexed, following the sub-steps:
determining the value Fₘ^{(art)}(|w_{m,n}^{(art)}|),
determining an intermediate value x verifying Fₘ^{(ref)}(x)=Fₘ^{(art)}(|w_{m,n}^{(art)}|),
setting the corrected coefficient w_{m,n}^{(corr)} equal to
$${w}_{m , n}^{( art )} \times min \{ 1 , \frac{x}{\vert {w}_{m , n}^{( art )} \vert} \} ,$$
determining a corrected signal using the discrete wavelet transform based on the corrected projection,
replacing in the signal the artifact signal by the corrected signal.

7. The method according to claim 1, comprising a determination of a minimum quantity of anesthetic to inject so that the subject remains in general anesthesia or sedation, the determination being based on the first parameter.

8. A general anesthesia or sedation assisting device comprising a processor configured for implementing the computer implemented method as claimed in claim 1.

9. A computer-readable medium comprising computer-executable instructions embodied which, when executed by a computer, cause the computer to carry out the method according to claim 1.
